(19) United States
(12) Patent Application Publication
(10) Pub. No.: US 2006/0165739 A1
(43) Pub. Date: Jul. 27, 2006

(54) Alcohol-free microemulsion composition

(75) Inventors: Napaporn Komesvarakul, Stratford, CT (US); James Faller, Williamsville, NY (US); Brian Jones, Flower Mound, TX (US); John Schiltz, Coppell, TX (US); Erika Szekeres, Pleasanton, CA (US); Anton Mentlik, Grapevine, TX (US); Louis Fisher, Dallas, TX (US); Gregg Nicoll, Atlanta, GA (US); David Sabatini, Norman, OK (US); John Scamehorn, Norman, OK (US)

Correspondence Address: FULBRIGHT & JAWORSKI L.L.P., 600 CONGRESS AVE., SUITE 2400, AUSTIN, TX, 78701, US

(73) Assignee: Mary Kay Inc., Addison, TX (US)

(21) Appl. No.: 11/325,605

(22) Filed: Jan. 3, 2006

Related U.S. Application Data
(60) Provisional application No. 60/642,217, filed on Jan. 6, 2005.
(60) Provisional application No. 60/667,454, filed on Apr. 1, 2005.
(60) Provisional application No. 60/669,089, filed on Apr. 7, 2005.

(51) Int. Cl. A61K 8/36

(52) U.S. Cl. 424/401

(57) ABSTRACT

The present invention concerns compositions that comprise alcohol-free microemulsions and methods for their use that include a surfactant, a lipophilic linker, and/or a hydrophilic linker. These compositions can be used, for example, in cosmetic or hair applications. In certain aspects, compositions of the invention have the ability to microemulsify sebum while providing enhanced cleansing of cosmetic products from the skin or hair. In addition, the compositions have the ability to enhance the penetration of skin or hair active ingredients, such as emollients, humectants, anti-oxidants, lipids, vitamins, botanicals, dyes, tanning compounds, etc.

This application claims the benefit of U.S. Provisional Application No. 60/642,217, filed Jan. 6, 2005, U.S. Provisional Application No. 60/667,454, filed Apr. 1, 2005, and U.S. Provisional Application No. 60/669,089, filed Apr. 7, 2005, the contents of which are incorporated into this specification by reference.

### BACKGROUND OF THE INVENTION

A. Field of the Invention

The present invention relates generally to alcohol-free microemulsions and methods for their use. The microemulsions can include a surfactant and a hydrophilic or lipophilic linker and can be used in a variety of cosmetic applications.

B. Background of the Invention

Microemulsion systems typically include oil, water, and a surfactant. These systems can form spontaneously and are therefore thermodynamically stable. The size of the droplets in such microemulsions typically ranges from 100-1000 angstroms (10-100 nm), and has very low oil/water interfacial tension. Because the droplet size is less than 25% of the wavelength of visible light, microemulsions appear transparent to the eye. There are several different types of microemulsion systems: (1) oil-in-water microemulsions wherein oil droplets are dispersed in the continuous aqueous phase; (2) water-in-oil microemulsions wherein water droplets are dispersed in the continuous oil phase; and (3) bi-continuous microemulsions wherein microdomains of oil and water are interdispersed within the system. In all three types of microemulsions, the interface is stabilized by an appropriate combination of surfactants and/or co-surfactants.

Microemulsions have been used in cosmetic cleansing applications because of their solvent properties and ability to remove oil from the skin. The typical microemulsion cleansing system, however, uses medium chain alcohols (see, e.g., PCT Application No. PCT/EP02/05977; Graciaa et al. 1992; U.S. Pat. No. 4,568,480. The use of such alcohols can be toxic or irritating to the skin. Alcohols can also dry the skin, creating unattractive flakes on the skin. Similarly, the microemulsification of larger amphiphilic oils such as triglycerides has been difficult to achieve (Huang and Lips 2004) and also use alcohols.

### SUMMARY OF THE INVENTION

The inventors have discovered novel compositions and alcohol-free microemulsions and methods for their use that can be used in a variety of aspects that are discussed throughout this document. The compositions of the present invention can include, for example, an alcohol-free microemulsion, the microemulsion comprising a surfactant and a lipophilic or a hydrophilic linker or both. The composition can be formulated into a cosmetic composition. In certain non-limiting aspects, the composition is capable of spontaneously microemulsifying, for example, a triglyceride, cholesterol, a fatty acid, sebum (including artificial and natural or human sebum), a C6-C40 molecule, lauric acid, oleic acid, isostearic acid, tricaprin, triolein, glycerol triisostearate, oleyl oleate, myristyl myristate, isostearyl isostearate, squalene, cholesterol oleate, or natural or synthetic oils (e.g. vegetable and animal oils). In certain aspects, the composition is comprised in a cosmetic vehicle. The cosmetic vehicle, for example, can include a cream, a lotion, a solution, an anhydrous base, a gel, or an ointment or any other vehicles discussed in this document or known to those of ordinary skill in the art.

In other embodiments, the microemulsion is a single bicontinuous phase of water and oil. The microemulsion can be transparent or semi-transparent. In other aspects, the microemulsion is an oil-in-water or a water-in-oil microemulsion. The microemulsion can also be a two phase system. The first phase can be predominately water and the second phase can be a water-in-oil microemulsion or an oil-in-water microemulsion. Alternatively, the first phase can be predominately oil and the second phase can be a water-in-oil microemulsion or an oil-in-water microemulsion. In other aspects, the microemulsion is a three phase microemulsion. In a three phase system, the first phase can be water, the second phase can be oil, and the third phase can be a single bicontinuous water and oil microemulsion, a water-in-oil microemulsion, or an oil-in-water microemulsion. In other non-limiting aspects, the microemulsions can be a Type I, II, III, or IV microemulsion or can transform from one type of emulsion to another type of emulsion.

The compositions of the present invention can be comprised in an anti-aging product, a moisturizing product, or cleansing product, or a pre-cleanser product. The composition can be adapted for application at least once, twice, three, four five, six, seven, eight, or more times a day during use.

The surfactants in the microemulsion can be any surfactant discussed throughout this document or known to those of ordinary skill in the art. Non-limiting examples include anionic surfactants, cationic surfactants, nonionic surfactants, amphoteric/zwitterionic surfactants, co-surfactants or mixtures thereof. Non-limiting examples of anionic surfactant include alkyl sulfosuccinate, sodium dioctyl sulfosuccinate (AOT), sodium dihexyl sulfosuccinate (AMA), ammonium or sodium lauryl ether sulfate, alkyl or acyl taurates, alkyl or acyl sarcosinates, alyl ether sulfates, alkyl ether sulfonates, or alkyl ether carboxylates (e.g., counterion can be sodium, ammonium, or potassium). Alkyl sulfosuccinate can include a mono or dialkyl sulfosuccinate or a C6-C22 sulfosuccinate. Non limiting examples of cationic surfactants include a quaternary ammonium compound (e.g., an alkyldimethylammonium haloginide), alkyl pyridinium chlorides or bromides, or other hydrogenides. Non-limiting examples of nonionic surfactants include lecithin, a Span group (e.g., Span 20, or 80), or a Tween group (e.g., Tween 20, 21, 40, 60, 60K, 61, 65, 80, 80K, 81, or 85), a sugar amide (e.g. polysaccharide amide), or an alkyl polyglucocide. Non-limiting examples of amphoteric surfactants include, for example, a quaternary amino acid, an alkyl amine oxide, or an alkyl betaine.

The lipophilic linkers that can be in the microemulsions can be any lipophilic linker that is discussed throughout this document or that is known to those of ordinary skill in the art. Non-limiting examples include glycerol monooleate, monoglyceride, an alkyl sorbital ester, a polyoxyethylene derivative of a sorbitan ester, or sorbitan isosterate (Crill 6). Monoglyceride can be glycerol monooleate, glycerol monostearate, glycerol monopalmitate, glycerol monomyristate, or glycerol monolaurate. Alkyl sorbital esters can include sorbitan monooleate (Span 80), sorbitan monostearate (Span 60), sorbitan monopalmitate (Span 40), sorbitan monolaurate (Span 20), or sorbitan trioleate (Span 85). Polyoxyethylene derivatives of a sorbitan ester can be POE (20) sorbitan monooleate (Tween 80) or POE (5) sorbitan monooleate (Tween 81).

The hydrophilic linkers that can be in the microemulsions can be any hydrophilic linker that is discussed throughout this document or that is known to those of ordinary skill in the art. Non-limiting examples include an alkyl glucoside, sodium mono or dimethyl naphthalene sulfonate (SMDMS), or sodium xylene sulfonate. Alkyl glucosides can be a hexyl, octyl, or decyl glucoside, for example.

In certain aspects, the compositions of the present invention comprise from about 0.1% to about 50% of the surfactant, from about 1.0% to about 40% of the surfactant, from about 5% to about 15% of the surfactant, about 10% of the surfactant or any range derivable therein (e.g. 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%). In other aspects, the compositions can include about 55%, 60%, 70%, 75%, 80%, 85%, 90%, 95% or of the surfactant. The compositions can also include from about 0.1% to about 50% of the lipophilic linker, from about 1.0% to about 40% of the lipophilic linker, from about 5% to about 20% of the lipophilic linker, or about 15% of the lipophilic linker or any range derivable therein (e.g. 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%). In other aspects, the compositions can include about 55%, 60%, 70%, 75%, 80%, 85%, 90%, 95% or of the lipophilic linker. The compositions of the present invention can include from about 0.1% to about 50% of the hydrophilic linker, from about 1.0% to about 40% of the hydrophilic linker, from about 5% to about 20% of the hydrophilic linker, or about 15% of the hydrophilic linker or any range derivable therein (e.g. 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%). In other aspects, the compositions can include about 55%, 60%, 70%, 75%, 80%, 85%, 90%, 95% or of the hydrophilic linker. In other embodiments, the compositions of the present invention comprise both a lipophilic and a hydrophilic linker. The compositions can include, for example, from about 0.1% to about 50% of the surfactant, from about 0.1% to about 50% of the lipophilic linker, and from about 0.1% to about 50% of the hydrophilic linker.

The compositions of the present invention can also include a co-oil. Co-oils that can be used with the present compositions can be any co-oil that is discussed throughout this document or that is known to those of ordinary skill in the art. Non-limiting examples include squalene, squalane, isopropyl myristate, ethyl laurate, artificial sebum, a cosmetic ester comprising from about a C6 to about a C30 group, or a compound comprising an equivalent alkane carbon number (EACN) similar to sebum, a mineral oil, a vegetable oil, an animal oil, oleyl oleate, chloresterol, glycerol tricaprylate, mineral oil, olive oil, almond oil, caprylic triglyceride, oleyl eructate, coco caprylate/caprate, or dioctyl cyclohexane. The compositions of the present invention can include from about 0.001% to about 30% of the co-oil, from about 1.0% to about 20% of the co-oil, from about 5% to about 15% of the co-oil, or about 10% of the co-oil or any range derivable therein (e.g. 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, or 29%). In other aspects, the compositions can include about 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 70%, 75%, 80%, 85%, 90%, 95% or of the co-oil.

The compositions of the present invention can also include a hydrotrope. Hydrotropes that can be used with the present compositions can be any hydrotrope that is discussed throughout this document or that is known to those of ordinary skill in the art. Non-limiting examples include an alkyl glucoside, sodium mono or dimethyl naphthalene sulfonate (SMDMS), sodium xylene sulfonate, or ammonium xylene sulfonate. Alkyl glucoside can be, for example, a hexyl, octyl, or decyl glucoside. A person of ordinary skill in the art will recognize that alkyl glucocides can be surfactants or hydrotropes depending on alkyl chain length. For example, decyl glucocide can also be a surfactant. The compositions of the present invention can include from about 0.001% to about 30% of the hydrotrope, from about 1.0% to about 20% of the hydrotrope, from about 2% to about 10% of the hydrotrope, or about 5% of the hydrotrope or any range derivable therein (e.g. 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, or 29%). In other aspects, the compositions can include about 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 70%, 75%, 80%, 85%, 90%, 95% or of the hydrotrope.

The compositions of the present invention can also be formulated to be chemically compatible. They can also further comprise water, oil, salts (non-limiting examples include NaCl, KCl, CaCl₂, or MgCl₂). In other aspects, the composition can also be used as a carrier for an active agent or a skin-active agent. Non-limiting examples of active agents include vitamins, minerals, humectants, emollients, anti-oxidants, oils, lipids, botanicals, tanning compounds, skin lightening compounds, UVA or UVB absorbers, sunscreens, infrared reflectors, infrared absorbers, or other agents that are discussed throughout this document and known to those of ordinary skill in the art.

The compositions of the present invention can readily absorb sebum into a single-phase, Type IV, microemulsion without the use of alcohol in the composition. The compositions can be used to provide superior cleansing of human sebum and cosmetic soils (foundations, mascara, colored cosmetics (eye shadow, cheek color, eye liners, etc.), moisturizers, etc. for skin types or hair represented by the general population.

In other non-limiting aspects, the compositions of the present invention can be used in pre-cleansing products, applications, or regimens. For example, the compositions of the present invention can be used to prepare the skin or hair prior to the application of a cosmetic product or hair product. As noted throughout this document, non limiting examples of cosmetic products include moisturizing creams, skin benefit creams, lotions, gels, ointments, foundations, night creams, lipsticks, cleansers, toners, masks, and/or other cosmetic products that are known to a person of ordinary skill in the art. Non-limiting examples of hair products include shampoos, conditioners, dyes, hairsprays, mousses, gels, hair detoxifier products, hair thickening products, hair texturizing products, hair shining or sheen products, hair volume products, hair growth products, hair repair products, products for oily, dry, brittle, or damaged hair (e.g., environmentally damaged hair), hair moisturizing products, hair products for thin or thinning hair, hair hydration products, or any other hair products that are known to a person of ordinary skill in the art.

In other aspects, the compositions can be suitable for extremely dry skin or hair. In addition to superior cleansing, the compositions of the present invention can be used to deliver active ingredients to the skin or hair. Non-limiting examples of active ingredients are discussed throughout this document are incorporated into this section by reference. By way of example only, the compositions of the present invention can deliver emollients or lipids to the skin barrier or hair to provide relief from dry skin or hair. The compositions of the present invention can also be tailored to be suitable for oily skin types or hair, and may provide superior sebum removal from deep within skin pores when used as a pre-cleanser or mask. The compositions can also be tailored to remove sebum from deep within the pores of oily skin, increasing the time it takes sebum to break through makeup and thus preventing shine for an extended period of time.

In another embodiment of the present invention, there is disclosed a method of cleaning skin or hair comprising applying to the skin or hair a composition comprising an alcohol-free microemulsion, the microemulsion comprising a surfactant and a lipophilic or a hydrophilic linker, wherein applying the composition cleans the skin or hair. The composition can, for example, spontaneously emulsify a triglyceride, or sebum (artificial or natural or human). The composition can spontaneously emulsify, for example, a triglyceride, cholesterol, a fatty acid, sebum (including artificial and natural or human sebum), a C6-C40 molecule, lauric acid, oleic acid, isostearic acid, tricaprin, triolein, glycerol triisostearate, oleyl oleate, myristyl myristate, isostearyl isostearate, squalene, cholesterol oleate, or natural or synthetic oils (e.g. vegetable and animal oils). The method can further include rinsing the skin with water to remove the composition. In certain aspects, the method is further defined as a method of absorbing sebum from the skin. The method can be further defined as a method of removing a cosmetic composition from the skin. The composition can spontaneously emulsify compositions of the present invention. Non-limiting examples of cosmetic compositions include mascara, foundation, eye shadow, lipstick, or eye liner. In other aspects, the method is further defined as a method of removing dirt or oil from the skin. The composition can spontaneously emulsify the dirt or oil. In certain aspects, the skin is facial skin.

There is provided another method of delivering an active agent to skin or hair comprising (a) applying a composition to the skin or hair, the composition comprising an alcohol-free microemulsion, the microemulsion comprising a surfactant, and a lipophilic or a hydrophilic linker or both, and (b) an active agent, wherein applying the composition to the skin or hair delivers the active agent to the skin or hair. The composition can be formulated as a cosmetic composition. Non-limiting examples of active agents include those discussed throughout this document and known to those of skill in the art, including vitamins, minerals, humectants, emollients, anti-oxidants, oils, lipids, botanicals, tanning compounds, skin lightening compounds, UVA absorbers, UVB absorbers, sunscreens, infrared reflectors, and infrared absorbers. In certain non-limiting aspects, the delivery of the active ingredient to the skin or hair is used to treat dry skin, oily skin, damaged hair (e.g. dry, brittle, oily, colored, etc.), or dirty or soiled hair. In other embodiments, the delivery of the active ingredient to the skin is used to improve the barrier properties of the skin. In other aspects, the composition spontaneously emulsifies a triglyceride, sebum (artificial or natural or human), or oil. The triglyceride, sebum, or oil can be the sebum, triglyceride, or oil that is on the skin or hair. The method can further include rinsing the skin or hair with water to remove the composition, dirt, or oil, for example.

In yet another aspect, there is provided a method of delaying the transmission of sebum through a cosmetic composition that is on skin comprising: (a) applying a composition comprising an alcohol-free microemulsion to the skin, the microemulsion comprising: (i) a surfactant; (ii) a lipophilic or a hydrophilic linker; and wherein applying the composition to the skin absorbs sebum from the skin, and (b) applying a cosmetic composition to the skin, wherein a reduction of sebum on the skin prior to topically applying the cosmetic composition delays the transmission of the sebum through the cosmetic composition that is subsequently applied to the skin. The cosmetic composition can be mascara, foundation, eye shadow, lipstick, eye liner, pressed powder, or loose powder. In other aspects, the composition can spontaneously emulsify a triglyceride, sebum, oil, dirt or other ingredients discussed throughout this document and known to those of skill in the art. The method can also include a further step of rinsing the skin with water to remove the composition.

In still another embodiment of the present invention, the disclosed alcohol-free microemulsions can be used in non-cosmetic applications such as oil-spill or clean-up applications. For instance, the alcohol-free microemulsions of the present invention can be used in a method of collecting oil upon or cleaning-up a surface, the method comprising dispensing a quantity of the alcohol-free microemulsion across the surface, wherein oil is absorbed by the microemulsion. Subsequently, the microemulsion can be removed from the surface. The microemulsion can be incorporated into a composition or material. In non-limiting embodiments, the composition or material can be any type of composition or material that a person of ordinary skill in the art would recognize as being useful in oil-spill or clean-up applications (e.g., cloth, paper-towels, washing materials and compositions (e.g., dish, shower, counter-top, or floor washing materials or compositions), hollow fibers, peat moss, polypropylene containing compositions, seaweed containing compositions, etc.). The surface, in non-limiting embodiments, can be water (e.g., ocean water, lake water, sea water, swimming pool water, etc.) or liquid surfaces or solid surfaces (e.g., counter-tops, dishware, ground, rocks, animals, machine parts, etc.). In non-limiting aspects, the oil to be removed can be petroleum-based, food-based, or human based oil.

“Damaged skin” and “damaged hair,” as those terms are used in the specification and claims, includes aged skin or hair, nutritionally compromised skin or hair, or environmentally damaged skin or hair. Environmentally damaged skin or hair includes, for example, skin or hair damaged by UV light, chronic sun exposure, environmental pollutants, chemicals, disease pathologies, or smoking.

The terms “mixture,” “mix,” and “mixing” or any variants of these terms, when used in the claims and/or specification includes, stirring, blending, dispersing, milling, homogenizing, and other similar methods. The mixing of the components or ingredients of the disclosed compositions can form into a solution. In other embodiments, the mixtures may not form a solution. The compositions can also exist as undissolved colloidal suspensions.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.” It is contemplated that any embodiment discussed in this specification can be implemented with respect to any method or composition of the invention, and vice versa. Furthermore, compositions of the invention can be used to achieve methods of the invention.

Throughout this application, the term “about” or “approximately” are used to indicate that a value includes the inherent variation of error for the device, the method being employed to determine the value, or the variation that exists among the study subjects. For instance, “about” or “approximately” are defined as being close to as understood by one of ordinary skill in the art, and in one non-limiting embodiment the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWING

The following drawings form part of the present specification and are included to further demonstrate certain aspects of the present invention. The invention may be better understood by reference to one or more of these drawings in combination with the detailed description of specific embodiments presented herein.

FIG. 1. Fish diagram showing phase behavior for sodium dihexyl sulfosuccinate and styrene as a function of surfactant concentration and salinity.

FIG. 2. Schematic of the linker concept, showing the surfactant (sodium dihexyl sulfosuccinate or SDHS), lipophilic linker (dodecanol), and hydrophilic linker (sodium mono- and dimethylnapthalene sulfonate or SMDNS).

FIG. 3. Fish diagram with squalene at 0.5% NaCl as a function of surfactant concentration and sebum fraction in oil.

FIG. 4. Fish diagram with squalane at 1.5% NaCl as a function of surfactant concentration and sebum fraction in oil.

FIG. 5. Fish diagram with squalane at different salinities as a function of surfactant concentration and sebum fraction in oil.

FIG. 6. Fish diagram with isopropyl myristate (IPM) at different salinities as a function of surfactant concentration and sebum fraction in oil.

FIG. 7. Fish diagram with ethyl laurate (EL) at different salinities as a function of surfactant concentration and sebum fraction in oil.

FIG. 8. Fish diagram with squalane and ethyl laurate (EL) at 1.5% NaCl as a function of surfactant concentration and sebum fraction in oil.

FIG. 9A-B. (A) Surfactant concentration versus sebum fraction in oil at 0.5% NaCl with squalene (volume of oil mixture is equal to volume of surfactant mixture). (B) Surfactant concentration versus sebum fraction in oil at 0.5% NaCl with squalene (volume of oil mixture is equal to volume of water containing in surfactant mixture, WOR=1).

FIG. 10A-B. (A) Surfactant concentration versus sebum fraction in oil at 0.5% NaCl with squalane (volume of oil mixture is equal to volume of water contained in surfactant mixture, WOR=1). (B) Surfactant concentration versus sebum fraction in oil at 1.5% NaCl with squalane (volume of oil mixture is equal to volume of water containing in surfactant mixture, WOR=1).

### DESCRIPTION OF ILLUSTRATIVE EMBODIMENTS

Aged, nutritionally compromised, and environmentally damaged skin affect many people. Fine lines, wrinkles, dry skin, loss of elasticity, increased sagging, loss of firmness, loss of color eveness, coarse surface texture, and mottled pigmentation are just some examples of the effects of damaged skin. People also use many skin cleaners in a variety of applications ranging from cosmetic, dirt, and oil removal. Skin cleaners can leave the skin feeling dry, irritated, and flaky. This is especially true of cleaners that include alcohol in the compositions.

Previous attempts to clean or treat damaged skin have various drawbacks ranging from skin irritation to skin toxicity. The present invention is an effective alternative to the use of microemulsion systems that use alcohol, hydroxy acids, retinoid compounds, or other materials currently used to clean or treat aged or environmentally-damaged skin.

The compositions and methods of the present invention can be used, e.g., in cleansing applications, to treat dry skin or damaged hair, to treat oily skin or hair, for reducing sebum breakthrough of cosmetic products, and for improving the skin or hair's visual appearance, function, and clinical/biophysical properties which have been changed by factors such as chronological age, chronic sun exposure, adverse environmental pollutants, household chemicals, disease pathologies, smoking, and malnutrition. In particular embodiments, the compositions include, e.g., an alcohol-free microemulsion comprising a surfactant; and a lipophilic or a hydrophilic linker. The composition can include a variety of other components ranging from co-oils, hydrotropes, salts, triglycerides, and skin-active agents. These and other aspects of the present invention are described in further detail throughout this document.

A. Microemulsions

In many cases a microemulsion is a smaller and thermodynamically stable form of an emulsion. An emulsion includes two immiscible phases (e.g., an oil phase and a water phase). In an oil-in-water emulsion, oil is dispersed in water, and oil forms a discontinuous phase and water forms a continuous phase. In a water-in-oil emulsion, water is dispersed in oil, and water forms a discontinuous phase and oil forms a continuous phase.

Besides being smaller than emulsions, microemulsions are also more thermodynamically stable (e.g., phase separation is prevented) and tend to appear more transparent or translucent than regular emulsions. This is because the interfacial tension between the two phases in the microemulsion is low; in some instances, lower than can be measured with conventional instruments such as a DuNouy Tensiometer. This low interfacial tension results from combinations of oil, surfactants, and water, and is related to the particle size of the dispersed phase being less than 1000 Angstroms. This size is relatively small in comparison to the wave length of visible light, thereby causing microemulsions to appear transparent. Microemulsions are thermodynamically stable and are stable toward phase separation.

Because of their oil-surfactant-water interface, microemulsions can form a variety of structures. In many instances, the size of these structures can be in the range of a few tens to hundreds of nanometers (e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, or 500 or more nanometers). The structures can include micelles (spherical or cylindrical objects formed by surfactant molecules, separating oil and water), lamellaes (water and oil consecutive layers separated by surfactant layers conveniently oriented), spherulite structures (onion structure), or bicontinuous structures (e.g., water and oil are continuous phases).

In certain embodiments of the present invention, the microemulsions form a single phase, Type IV microemulsion (bicontinuous phase of water and oil). Other microemulsions that can form include: Type I microemulsions (2 phase, oil-in-water) which can be visualized by swollen micelles surrounded by water where surfactant micelles coexist with excess oil; Type II microemulsions (2 phase, water-in-oil) which can be visualized as swollen reversed micelles surrounded by oil where the reversed micelles coexist with excess oil; and Type III microemulsions (3 phase systems) which corresponds to an oil, water, and a middle bicontinuous microemulsion phase coexisting in a three-phase equilibrium. Microemulsions have the ability to allow the mixing of water and oil in a thermodynamically stable state without the use of mechanical agitation to produce the single-phase solution.

Microemulsion transition can be achieved in several ways, depending on the type of surfactant. In certain instances, for example, the different microemulsion types (Type I, II, III, and IV) can be formed by varying the fraction of co-oil in the oil mixture and/or salt in the mixture. A Type IV microemulsion, for example, has been observed at high surfactant/linker concentrations. In other aspects, for instance, in ionic surfactant systems, a Type I-III-II transition can be obtained by increasing electrolyte concentration whereas increasing temperature can achieve the same transition for nonionic surfactant systems. The electrolyte concentration required at the optimum condition is called “optimum salinity” or S*. The optimum condition includes a condition at which an equal volume of oil and water is solubilized in the bicontinuous phase (Type III). The Solubilization parameter (SP), which is defined by the amount of oil solubilized in the middle phase per unit mass of surfactant, at this optimum conditions is then called optimum solubilization parameter (SP*).

Microemulsions of the present invention may be prepared by a number of methods known to those skilled in the art (see, e.g., U.S. Pat. No. 4,146,499). For example, the microemulsions can be formed from the components discussed throughout this document with the aid of a stirrer or blending equipment. Other commercially available emulsifying equipment providing mechanical agitation can also be used to prepare the microemulsions.

The microemulsions of the present invention can include, for example, surfactants, lipophilic linkers, hydrophilic linkers, hydrotropes, co-oils, salts, and other ingredients that are known to those of ordinary skill in the art and that are described in more detail throughout this document.

B. Surfactants

The term surfactant is derived from the phrase surface active agent. Surfactants are typically amphiphilic molecules that can be absorbed at various interfaces and can change the properties of the interfaces. Surfactants have wide range applications from oil recovery, cleansing applications, to efficient delivery of drugs at a desired site in the body.

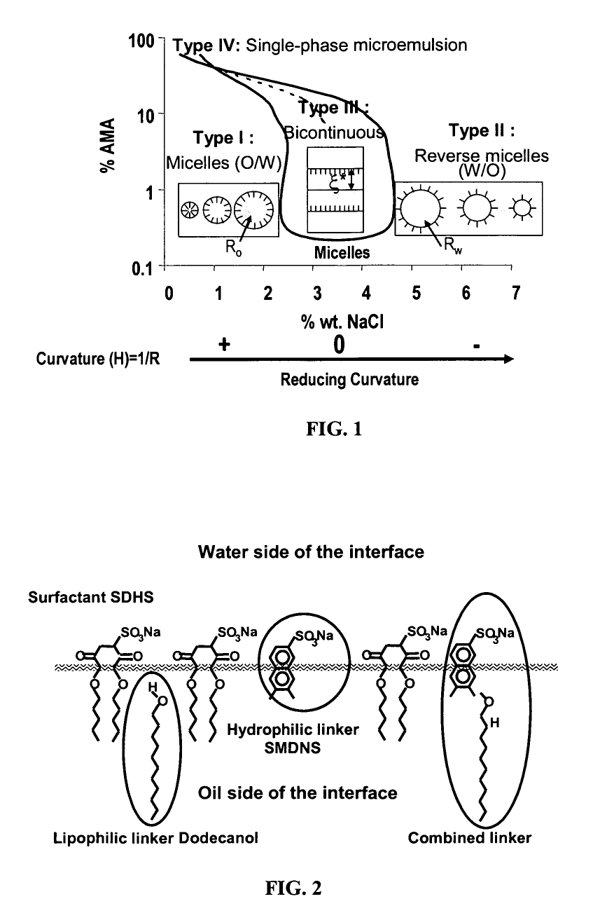

There are two important parameters which describe the ability and effectiveness of a surfactant to form microemulsions, the spontaneous curvature and the flexibility of the surfactant film it forms (Daicic et al., 1995). The curvature of the surfactant film depends both on the nature of the surfactant and on the composition of the polar and nonpolar phases. An elastic and flexible surfactant film favors the formation of a microemulsion, whereas a lamellar phase is formed with a more rigid or stiff film. The flexibility of the film also depends on the molecular structure of the surfactant and can be reduced by the addition of co-solvents such as short chain alcohols (Von Corswant et al., 1997; Von Corswant et al., 1998a; Von Corswant et al., 1998b, Von Corswant et al. 1998c). While microemulsion phase behavior of microemulsion systems can be described in various ways, the “fish diagram” is one of the most common. A fish diagram is typically plotted between surfactant concentration and a scan parameter or a tuning parameter (e.g. salt or hydrophobicity of the system), as shown in FIG. 1. The scan parameter directly affects the curvature of the surfactant membrane which is a factor for a surfactant to form microemulsions.

The curvature is defined as positive when the film curves around the oil and is negative when the film curves around the water, as shown in FIG. 1. Addition of electrolyte into surfactant systems increases surfactant hydrophobicity and decreases the surfactant film curvature. Therefore, when the surfactant system has relatively low hydrophobicity or is at low salinity, a Type I microemulsion (O/W microemulsion) can occur. At high hydrophobicity where the curvature decreases, a Type II microemulsion (W/O microemulsion) can exist. While intermediate between these two conditions and at lower surfactant concentration, the three-phase microemulsion or Type III microemulsion can occur with a net zero curvature. When the surfactant concentration increases above Type III region, a Type IV microemulsion can be obtained. The minimum surfactant concentration for complete solubilization of the water and the oil is where the three-phase and one-phase regions (Type IV) meet, which appears at relatively high surfactant concentrations.

The alcohol-free microemulsions of the present invention can include a surfactant or multiple surfactants. Surfactants that can be used with the present invention can be natural or synthetic and can be cationic, anionic, zwitterionic, nonionic, or mixtures thereof (Rosen 1988; Rieger 1999). U.S. Pat. No. 6,495,126, for example, provides a non-limiting list of the different types of surfactants that can be used with the present invention. In certain non-limiting embodiments, for example, the surfactant can be sodium dioctyl sulfosuccinate (AOT) or sodium dihexyl sulfosuccinate (AMA). The chemical structure of AOT, for example, is:

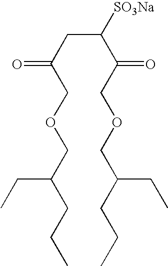

Suitable cationic surfactants include, but are not limited to, DMDAO or other amine oxides, long-chain primary amines, diamines and polyamines and their salts, quaternary ammonium salts, polyoxyethylenated long-chain amines, and quatemized polyoxyethylenated long-chain amines.

Non-limiting examples of anionic surfactants include SDS, salts of carboxylic acids (i.e. soaps), salts of sulfonic acids, salts of sulfuric acid, phosphoric and polyphosphoric acid esters, alkylphosphates, monoalkyl phosphate (MAP), and salts of perfluorocarboxylic acids.

Examples of zwitterionic surfactants include, but are not limited to, cocoamidopropyl hydroxysultaine (CAPHS) and others which are pH-sensitive and require special care in designing the appropriate pH of the formula (i.e. alkylaminopropionic acids, imidazoline carboxylates, and betaines) or those which are not pH-sensitive (i.e. sulfobetaines, sultaines).

Suitable nonionic surfactants can include, but are not limited to, alkylphenol ethoxylates, alcohol ethoxylates, polyoxyethylenated polyoxypropylene glycols, polyoxyethylenated mercaptans, long-chain carboxylic acid esters, alkonolamides, tertiary acetylenic glycols, polyoxyethylenated silicones, N-alkylpyrrolidones, and alkylpolyglycosidases.

In other embodiments, any combination of the surfactants discussed in this document or known to a person of skill in the art is also acceptable. For example, a surfactant can include at least one anionic and one zwitterionic surfactant, or at least one anionic and one nonionic surfactant which are compatible.

C. Lipophilic and Hydrophilic Linkers

Linkers of the present invention can be used to augment the interaction between the surfactant and oil phase (lipophilic linkers) or between the surfactant and water phase (hydrophilic linkers). Lipophilic or hydrophilic linkers or both in combination can be used to increase the solubilization capacity in microemulsions several-fold (Graciaa et al. 1993).

FIG. 2 provides a schematic of the linker concept. Lipophilic linkers tend to segregate near the oil side of oil/water interface close to the tails of the surfactants (Acosta et al., 2003). In FIG. 2, the surfactant, sodium dihexyl sulfosuccinate, adsorbs at the oil/water interface. The lipophilic linker, dodecanol, is shown to adsorb at the palisade layer of the interface (oil side of the surfactant layer), promoting the local order and increasing the interaction between surfactant tail and the oil phase. Sodium mono and dimethylnaphthalene sulfonate (SMDNS) is a hydrophilic linker which adsorbs on the water side of the oil/water interface. This hydrophilic linker molecule is believed to increase the total interfacial area and the overall interaction between the surfactant layer and the aqueous phase (Acosta et al., 2002). Adding lipophilic linker alone to microemulsions gives limited solubilization enhancement. Hydrophilic linkers can help improve solubilization ability because they allow more room for lipophilic linkers to segregate and further enhance the solubilization ability (Acosta et al., 2003; Acosta et al., 2002a; Acosta et al., 2002b).

The alcohol-free microemulsions of the present invention can include a lipophilic or a hydrophilic linker or both. Natural and synthetic linkers can be used with the present invention. Non-limiting examples of lipophilic linkers include monoglycerides such as glycerol monooleate (GMO), glycerol monostearate, glycerol mono palmitate, glycerol monomyristate, and glycerol monolaurate; alkyl sorbital esters such as sorbitan monooleate (Span 80), sorbitan monosterate (Span 60), sorbitan monopalmitate (Span 40), sorbitan monoluarate (Span 20), and sorbitan trioleate (Span 85); polyoxyethylene derivatives of sorbitan esters such as POE (20) sorbitan monooleate (Tween 80) and POE (5) sorbitan monooleate (Tween 81); and sorbitan isosterate (Crill 6). The chemical structure of Span 80, for example, is:

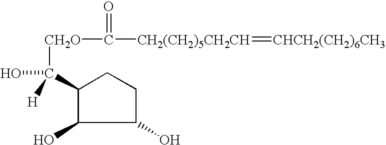

Non limiting examples of hydrophilic linkers that can be used with the present invention include alkyl glucosides (e.g., hexyl, octyl, decyl glucosides), sodium mono and dimethylnaphthalene sulfonate (SMDNS), and sodium xylene sulfonate. The chemical structure of hexyl glucoside, for, example, is: 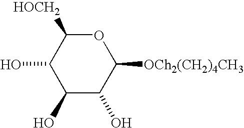
D. Co-Oils

The alcohol-free microemulsions of the present invention can include a natural or synthetic co-oil. Non-limiting examples of co-oils include squalene, squalane, isopropyl myristate, ethyl laurate, artificial sebum, cosmetic esters with components from C6 to C30, and compounds having an equivalent alkane carbon number (EACN) close to sebum (approximately =13), ranging from 3 to 35. The chemical structures of squalene (EACN=24 and MW=410), squalane (EACN=˜24 and MW=422), isopropyl myristate (EACN=13 and MW=270), and ethyl laurate (EACN<13 and MW=224), for example, are: 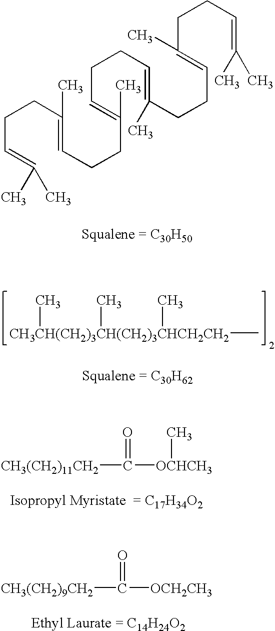
E. Hydrotropes

Hydrotropes are organic substances that can increase the solubility of other organic substances in water. Hydrotropes, for example, can be used in the present invention in certain embodiments to stabilize surfactants, thereby allowing the surfactants to remain soluble.

The alcohol-free microemulsions of the present invention can include a natural or synthetic hydrotrope. Non-limiting examples of hydrotropes include ammonium xylene sulfonate, sodium xylene sulfonate, sodium mono- and di-methyl naphthalene sulfonate (SMDNS), and alkyl glucosides (e.g. hexyl, octyl, decyl glucosides).

F. Source of Components and Compounds

The specific components, compounds, and active ingredients that are contemplated as being used in the compositions and methods of the present invention can be obtained by any means known to a person of ordinary skill in the art. For example, the components, compounds, and active ingredients can be isolated by obtaining the source of such compounds. The compounds and active ingredients can be purified by any number of techniques known to a person of ordinary skill in the art. Such purification techniques include, e.g., Polyacrylamide Gel Electrophoresis, High Performance Liquid Chromatography (HPLC), Gel chromatography or Molecular Sieve Chromatography, and Affinity Chromatography.

In addition, the components, compounds, and active ingredients can be obtained by chemical synthesis or by recombinant means by using conventional techniques. For example, various automatic polypeptide synthesizers are commercially available and can be used in accordance with known protocols. See, for example, Stewart and Young, (1969); Tam et al., (1983); Merrifield, (1986); and Barany and Merrifield (1979), Houghten (1985).

G. Equivalents

Known and unknown equivalents to the specific compounds, components and active ingredients discussed throughout this document can be used with the compositions and methods of the present invention. The equivalents can be used as substitutes for the specific compounds, and active ingredients. The equivalents can also be used to add to the methods and compositions of the present invention. A person of ordinary skill in the art would be able to recognize and identify acceptable known and unknown equivalents to the specific compounds, extracts, and active components in such compounds and extracts without undue experimentation.

H. Compositions of the Present Invention

A person of ordinary skill would recognize that the compositions of the present invention can include any number of combinations of components, compounds and active ingredients such as, for example, surfactants, lipophilic linkers, hydrophilic linkers, hydrotropes, co-oils, and salts that are described in more detail below and throughout this document. It is also contemplated that that the concentrations of these compounds can vary. In other non-limiting embodiments, for example, the compositions may include in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein of at least one of the compounds (e.g., surfactants, hydrophilic and lipophilic linkers, co-oils, or hydrotropes), skin active ingredients, or derivatives that are mentioned throughout the specification and claims. In non-limiting aspects, the percentage can be calculated by weight or by volume of the total weight or volume of the composition. A person of ordinary skill in the art would understand that the concentrations can vary depending on the addition, substitution, and/or subtraction of the compounds and skin active ingredients and acceptable substitutes.

The disclosed compositions of the present invention may also include various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

I. Cosmetic Vehicles

The present compositions are effective in all types of cosmetic vehicles. Non-limiting examples of suitable cosmetic vehicles include emulsions, creams, lotions, solutions, anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the present invention.

J. Cosmetic Products

The composition of the present invention can also be used in many cosmetic products including, but not limited to moisturizing cream, skin benefit creams and lotions, gels, ointments, foundation, night cream, lipstick, cleansers, toners, masks, and/or other cosmetic products that are known to a person of ordinary skill in the art. In certain aspects, the composition of the present invention is preferably used in cleansing products for the face and other body parts.

K. Additional Compounds and Agents that can be Used in Combination with the Present Compositions

Compositions of the present invention can include other beneficial agents and compounds such as, for example, acute or chronic moisturizing agents (including, e.g., humectants, occlusive agents, and agents that affect the natural moisturization mechanisms of the skin), anti-oxidants, sunscreens having UVA and/or UVB protection, skin lightening agents (e.g. hydroquinone), emollients, thickeners (e.g., fused silica), anti-irritants, vitamins, trace metals, anti-microbial agents, botanical extracts, fragrances, and/or dyes and color ingredients (e.g., dyes, lakes, etc.).

1. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus arneniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays)oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquatemium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

2. Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCI, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

3. Compounds Having Ultraviolet Light Absorbing Properties

Non-limiting examples of compounds that have ultraviolet light absorbing properties that can be used with the compounds of the present invention include benzophenone, benzophenone-1, benzophenone-2, benzophenone-3, benzophenone-4 benzophenone-5, benzophenone-6, benzophenone-7, benzophenone-8, benzophenone-9, benzophenone-10, benzophenone-11, benzophenone-12, benzyl salicylate, butyl PABA, cinnamate esters, cinoxate, DEA-methoxycinnamate, diisopropyl methyl cinnamate, ethyl dihydroxypropyl PABA, ethyl diisopropylcinnamate, ethyl methoxycinnamate, ethyl PABA, ethyl urocanate, glyceryl octanoate dimethoxycinnamate, glyceryl PABA, glycol salicylate, homosalate, isoamyl p-methoxycinnamate, PABA, PABA esters, Parsol 1789, and isopropylbenzyl salicylate.

4. Additional Compounds and Agents

Non-limiting examples of additional compounds and agents that can be used with the compositions of the present invention include skin lightening agents (e.g. kojic acid, hydroquinone, ascorbic acid and derivatives, retinoids and their derivatives, and niacinamide), emollients (e.g. esters and fatty acids), vitamins (e.g. D, E, A, K, and C), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), preservatives (e.g. BHA), emollients (i.e. organic esters, fatty acids, lanolin and its derivatives, plant and animal oils and fats, and di- and triglycerides), antimicrobial agents (e.g., triclosan and ethanol), and fragrances (natural and artificial).

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Procedures

The inventors have developed compositions comprising alcohol-free microemulsions and methods for their use. The following includes non-limiting materials and methods used in one aspect of the present invention. It will be appreciated by a person of ordinary skill in the art that the materials used in the following examples can be substituted, added to, or subtracted from the compositions of the present invention. Additionally, the methods used to determine the effectiveness of the compositions of the present invention are non-limiting aspects, and it is contemplated that other methods known to those of ordinary skill in the art can be used.

Materials: The following materials were obtained from Aldrich (Milwaukee, WI) at the concentrations shown and were used without further purification: sorbitan monooleate (Span 80, 99%), squalene (98%), squalane (99%), isopropyl myristate (IPM, 98%), ethyl laurate (99%), and sodium chloride (99%). Sodium dioctyl sulfosuccinate (AOT, ˜100%) was purchased from Fisher Scientific (Fair Lawn, N.J.). Hexylpolyglucocide AG ₆₂₀₆™, donated by Akzo Nobel (Chicago, Ill.), was received as a 75% wt. aqueous solution and used without further purification. Artificial sebum was prepared by Mary Kay Inc. with the composition shown in Table 1. Properties of the co-oils are shown in Table 2.

TABLE 1
Composition of Artificial Sebum

| Components | % |
|---|---|
| Lauric acid | 11.73% |
| Oleic acid | 11.73% |
| Isostearic acid | 5.86% |
| Tricaprin | 11.73% |
| Triolein | 11.73% |
| Glycerol triisostearate | 5.86% |
| Oleyl oleate | 10.6% |
| Myristyl myristate | 10.6% |
| Isostearyl isostearate | 4.13% |
| Squalene | 12.23% |
| Cholesterol | 1.53% |
| Cholesterol oleate | 2.27% |

TABLE 2
Properties of Non-limiting Co-oils

| Co-Oil | EACN | MW (g/mole) | Molecular Formular |
|---|---|---|---|
| Squalene | 24 | 410 | 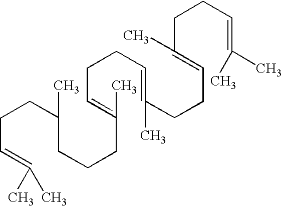 |
| squalane | ˜24 | 422 | 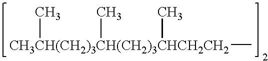 |
| Isopropylmyristate (IPM) | 13 | 270 | 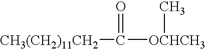 |
| Ethyllaurate (EL) | <13 | 224 | 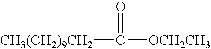 |

Methods: Phase behavior studies were performed using equal volume of water and oil (or sebum/co-oil mixtures), giving a water/oil ratio (WOR) equal to one. Preliminary studies were conducted to determine preferred formulations and salinity. In one non-limiting aspect, a preferred formulation of the aqueous phase was found to be a surfactant mixture of 4% AOT+5.13% sorbitan monooleate+5.06% hexylglucocide by weight. In another non-limiting aspect, a preferred salinity (S*) for this composition was 0.5% NaCl. As noted throughout this document and contemplated by the inventors, these concentrations can be varied. Additionally, the ingredients can also vary. For example, the surfactants, hydrophobic and hydrophilic linkers, co-oils, and hydrotropes that are discussed throughout this document and known to those of ordinary skill in the art can be used with the present invention.

Stock solutions of AOT, hexylglucocide, and sorbitan monooleate at the selected weight ratio were prepared at different total surfactant/linker concentrations ranging from 14.19% to 56.06% wt. The phase studies were carried out in 16×125 mm flat-bottomed tubes; 2.5 mL surfactant solution was added, followed by the addition of co-oil, then sebum oil. The total volume of co-oil and sebum oil required is equivalent to the amount of water present in the 2.5 mL surfactant solution; this is to keep WOR equal to one. The fraction of sebum oil in the oil mixtures is varied from zero (100% vol. co-oil) to one (100% vol. sebum oil). The prepared samples were gently shaken once a day for 3 days and left to equilibrate at room temperature for 2 weeks. Phase diagrams (fish diagrams) were constructed by plotting the total surfactant/linker concentration as a function of the sebum fraction in oil. The microemulsion phase (Type I, II, III, and IV) were obtained by visual observation. The effect of salinity (0.5%, 1.5%, and 3% wt. NaCl) and co-oil on the phase behavior was investigated.

### Example 2

### EACN of Co-Oils and Salinity

Formulating microemulsions requires the combination of variables that will provide a middle phase microemulsion. Salager et al. (1979) proposed a semi empirical equation that relates the different formulation variables:
Ln(S*)=k(EACN)+f(A)−σ+αTΔT
Where S* is an optimum salinity, or electrolyte concentration; k is a constant, normally, between 0.1 to 0.17, and EACN is the equivalent alkane carbon number for nonlinear hydrocarbon (e.g. triglycerides). Although preferred salinity concentrations may exist, the inventors contemplate that salinity levels can and will vary. For example, a person of ordinary skill in the art will recognize that salinity levels may vary depending on the desired effects of a given product, protocol, or individual characteristics of a user of the product. For linear hydrocarbon, alkane carbon number (ACN) is applied. The EACN is estimated based on the optimum salinity obtained in the inventors' formulation studies. The higher the optimum salinity, the more the hydrophobicity or EACN of the oil. The effect of alcohol or additives is noted by f(A), σ is a function of the type of the surfactant, α is a constant, and T is the temperature of the system. However, in this study, alcohol is not included and the temperature of the system is constant.

The EACN of squalene is 24 and isopropyl myristate (IPM) is 13. The EACN for squalane is expected to be close to the value for squalene (˜24). Table 3 shows the optimum salinity of oil mixtures (co-oil and sebum mixtures).

TABLE 3
Optimum Salinities for Oil Mixtures

| Isopropyl Myristate (IPM) | | | Ethyl Laurate (EL) | | |
|---|---|---|---|---|---|
| % IPM | % Sebum | S* | % EL | % Sebum | S*, % |
| 20 | 80 | <0.5 | 20 | 80 | <0.5 |
| 40 | 60 | 0.5 | 40 | 60 | <0.5 |
| 60 | 40 | 0.8 | 60 | 40 | <0.5 |
| 80 | 20 | 1.5 | 80 | 20 | 0.5-0.7 |
| 100 | 0 | 3.5 | 100 | 0 | 1-1.5 |

A preferred salinity of pure isopropyl myristate for this non-limiting aspect of the invention is 3.5% NaCl whereas the preferred salinity is lowered when the amount of sebum oil is increased (e.g. the optimum salinity for the oil mixture of 20% vol. IPM and 80% vol. sebum is less than 0.5%). This suggests that IPM has a higher EACN or is more hydrophobic than sebum oil. The preferred salinity of pure ethyl laurate (EL) is 1-1.5% NaCl, which is closer to the preferred salinity of a 20% vol. EL and 80% vol. sebum mixture, indicating that EL has a closer EACN to sebum oil than IPM does (EACN_{sebum}<EACN_{EL}<EACN_{IPM}).

This finding is significant in formulating cleansing products because the amount of sebum produce on human skin or hair can be different depending on skin or hair types. The ideal objective is to be able to formulate a product that is robust over a wide range of sebum oil secretion rates. A non-limiting strategy is finding a co-oil that has a similar EACN to the sebum oil, resulting in a similar optimum salinity. Therefore, based on this study, ethyl laurate is a preferred co-oil. However, the co-oils described throughout this document and those known in the art are contemplated as being useful with the present invention.

### Example 3

### Effect of Sebum Fraction in Oil and Surfactant Concentrations on Phase Diagram

Microemulsion phase transition: A Fish diagram for the system with squalene as co-oil at 0.5% NaCl is shown in FIG. 3. The fish tail is observed in the high concentration regime whereas the fish body appears in the low concentration regime. A surfactant concentration 14.19% wt was studied. As discussed throughout this document, however, the surfactant concentration can be varied. At lower surfactant concentrations, slow phase separation kinetics made it difficult to map out the three phase region. The surfactant concentration and the sebum fraction of oil at which the body and tail of the fish meet are denoted by “C” and “F”, respectively. The concentration C for this system, approximately, is 25% wt. surfactant concentration at the fraction F of 0.4, as summarized in Table 4.

TABLE 4
Concentration (C) and sebum fraction in oil (F) in a non-limiting Type IV
microemulsion

| | [NaCl], % | | | | | |
|---|---|---|---|---|---|---|
| | C | | | F | | |
| Oil | 0.50% | 1.50% | 3.00% | 0.50% | 1.50% | 3.00% |
| Squalene | 25 | | | 0.45 | | |
| Squalane | 25 | 35 | 42 | 0.35 | 0.2 | 0.15 |
| IPM | 25 | 38 | | 0.5 | 0.175 | |
| EL | 25 | 38 | | 0.4 | 0.02 | |

When the fraction of sebum in oil is zero, a Type I microemulsion forms. The presence of a co-oil aids microemulsion formation. Without co-oil (when sebum fraction is equal to one), a Winsor Type I forms at high surfactant concentration and no microemulsion forms at lower surfactant concentrations. When the sebum fraction in the oil increases, a Winsor Type I-III-II transition is observed at a low surfactant concentration regime up to 25% total surfactant concentration. A Winsor Type I-IV-I transition appears at high surfactant concentrations although at intermediate surfactant concentrations Winsor Type I-IV-III and I-IV-II transitions occur with an increase in sebum fraction in the oil.

The artificial sebum is comprised of several compounds as shown in Table 1, above. Almost one-third of the sebum is fatty acids which contribute to the greater hydrophilicity of the sebum oil, compared to triglycerides. Squalene is a long chain hydrocarbon oil that is present in the artificial sebum. Using squalene as co-oil in the microemulsion-based formulation can provide an efficient environment for the complicated comb-like structured triglycerides, enhancing the solubilization ability for artificial sebum. The co-oil can tune the spontaneous curvature of the surfactant monolayer and the addition of co-oil also increases the flexibility of the surfactant film, similar to the effect of adding a short chain alcohol (Von Corswant et al., 1997; Von Corswant et al., 1998b). Both effects are due to an increased interaction of squalene with the hydrocarbon region of the surfactant system, leading to a high degree of interaction between triglyceride and the nonpolar part of the surfactant film. The explanation is further supported by the fact that the co-oil can be microemulsified with the surfactant system. As seen in FIG. 3, at low surfactant concentration, no microemulsion forms without the presence of squalene.

Squalene is a nonpolar oil which is relatively hydrophobic, compared to the sebum oil. Therefore, microemulsification of squalene alone may use a more hydrophobic surfactant system. When squalene is present alone (without sebum oil), a Type I microemulsion is observed. This suggests that the surfactant system is relatively hydrophilic, resulting in a positive curvature of the surfactant film with the oil droplets. A Type I-III-II transition can be achieved in at least two ways: increasing the hydrophobicity of the surfactant system (aqueous phase) or increasing hydrophilicity of the oil. Increasing the hydrophobicity of the surfactant system helps move the surfactant system to aqueous phase/oil phase interface, whereas increasing hydrophilicity of the oil phase helps match the hydrophicity of the oil phase to the aqueous phase. The hydrophobicity/hydrophilicity matching leads to an increase in penetration of the surfactant film into the oil phase, a decrease in the curvature from positive values (Type I) to negative values (Type II), and an increase in the flexibility of the film. As seen in these results, the Type I-III-II transition is obtained when the fraction of sebum in oil increases (oil hydrophilicity increases). The addition of sebum oil to the system induces a change in the microstructure of the microemulsion from an O/W type to an O/W type.

This suggests that the interaction between surfactant film and the sebum oil is increased as the hydrophilicity of the oil mixture increases. In some non-limiting instances, less than 30% surfactant is necessary to microemulsify triglyceride based oil at room temperature. This is a surprising and unexpected result because previous reports indicated that up to 50% surfactants and co-surfactants for triglyceride microemulsification and at higher temperature (Von Corswant et al., 1998a; Tungsubutra and Miller, 1994) is needed. The required temperature in the microemulsification of sebum oil is also much lower than the temperatures that were reported when studying with triolein. This is attributed to the presence of fatty acids in the sebum which facilitate the oil solubilization (Huang and Lips, 2004; Tungsubutra and Miller, 1994).

Fish diagrams for the systems with squalane, isopropyl myristate, and ethyl laurate at 0.5% NaCl show similar behavior to the results shown in FIG. 3; therefore, detailed description of these systems is not provided. Von Corswant et al. (1998b) have found that adding isopropyl myristate into microemulsions based on triglycerides decreased the spontaneous curvature of the surfactant film and increased flexibility of the surfactant monolayer. The change in spontaneous curvature was manifested by a gradual change in the microstructure of the microemulsion, as revealed by NMR self-diffusion data (Von Corswant et al., 1997; Von Corswant et al., 1998b). A Type I-III-II transition for a long chain triglyceride was observed when the amount of EPM increase whereas an opposite trend is observed here. That is, a Type I-III-II transition occurs when the sebum fraction in oil increases or when the fraction of IPM in the oil mixture decreases. This might be due to the fact that the surfactant that Von Corswant et al. used, which is soybean phosphatidylcholine (SbPC), is relatively hydrophobic, so when the oil mixture is relatively hydrophobic, the degree of surfactant-oil interaction increases. This is decreases the curvature and increases in the flexibility of the film, inducing a Type I-III-II transition. The microemulsion system water/1-propanol/SbPC/EPM forms W/O microemulsion (that is, the spontaneous curvature of the SbPC film is negative). In the system reported here, the increased hydrophilicity of the sebum and co-oil mixture surprisingly and unexpectedly enhances the surfactant-oil interaction, leading to a Type I-III-II transition as well when the amount of IPM present is reduced. In other words, the spontaneous curvature for the surfactant film investigated here is positive when co-oil is present alone.

Surfactant partitioning at the excess water/middle phase and the middle phase/excess oil interfaces: When surfactant concentrations (y-axis) are plotted as a function of a tuning parameter such as salinities or hydrophobicity (x-axis), a fish diagram typically appears to be vertical in both fish body and fish tail. This suggests an insignificant partitioning of lipophilic and hydrophilic compounds from a bicontinuous middle phase into an excess oil phase and an excess water phase, respectively. In some cases, the head of the fish can slant towards lower salt concentration when the surfactant concentration decreases. This can be interpreted that as the surfactant concentration increases, the middle phase microemulsion requires higher salinities; or the surfactant system in the middle phase becomes more hydrophilic, suggesting that the lipophilic compound present in the middle phase partitions into the excess oil. In contrast, if the head of the fish slants towards higher salt concentrations, the middle phase becomes more hydrophobic and the partitioning of hydrophilic compound into the excess water can be expected. This phenomenon was initially mentioned by Bourrel and Schecter (1988). As shown in FIG. 3, the fish leans towards high hydrophobicity oil when the sebum fraction in oil is close to zero or when the fraction of co-oil is equal to one. In other words, when surfactant concentration increases, the middle phase microemulsion uses more hydrophilic oil. This translates that the middle phase becomes more hydrophilic, suggesting the partitioning of the lipophilic compound, which is sorbitol monooleate, into the excess oil phase.

### Example 4

### Effect of Salinity on Fish Diagram for Squalane, IPM, and EL

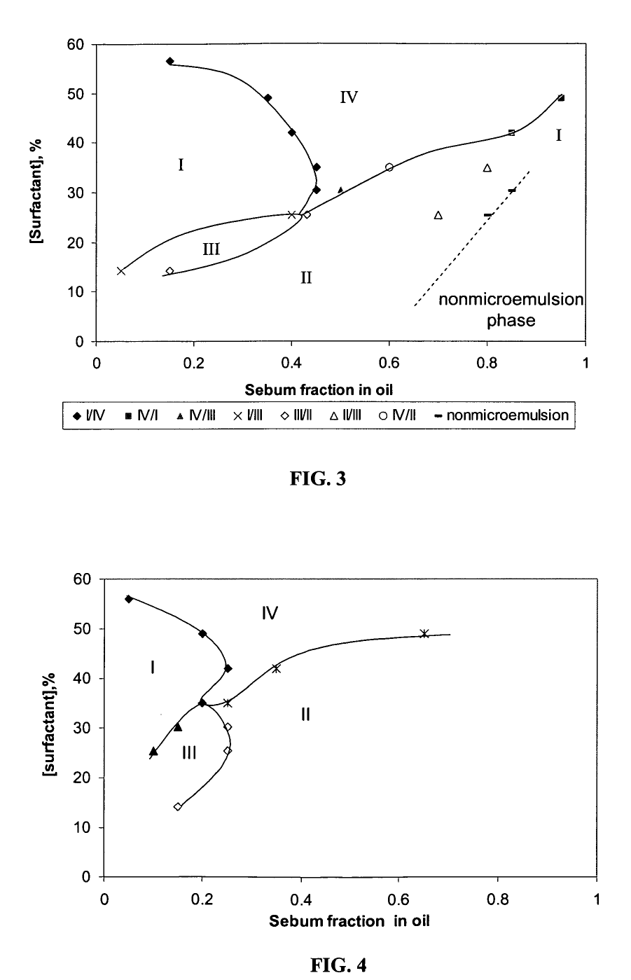

A fish diagram with squalane at 1.5% NaCl is shown in FIG. 4. The fish diagram at this salt concentration, compared to the fish diagram at 0.5% salt concentration (FIG. 3), is slightly different. At 1.5% NaCl, Winsor I-III-II and I-IV-II transitions are observed at low and high surfactant concentrations, respectively. These Winsor I-III-II and I-IV-II transitions are also observed at 1.5% NaCl for the systems with IPM and EL as co-oil as well as at 3.0% NaCl for the system with squalane.

For the system at 0.5% NaCl, Winsor I-IV-I is observed at high surfactant concentration, as shown in FIG. 3. As mentioned earlier, the phase behavior at 0.5% NaCl for squalene and squalane are similar so one can qualitatively compare the phase behavior for the system at low salt concentration (studied squalene) to the phase behavior at high salt concentrations (studied with squalane).

For the system at 1.5% NaCl, the system becomes hydrophobic by adding salt, and a Type II microemulsion is expected to be formed. For the system at 0.5% NaCl, as illustrated by the slanted fish in FIG. 3, when the surfactant concentration increases, the surfactant system in the middle phase becomes more hydrophilic as more hydrophilic oil is used (the fish head slants towards less hydrophilic oil). This suggests that the surfactant system has a higher salinity at higher surfactant concentrations to push the system towards the Type III or II microemulsions. Because the salt concentration is constant at 0.5% NaCl, a Type I microemulsion is observed instead.

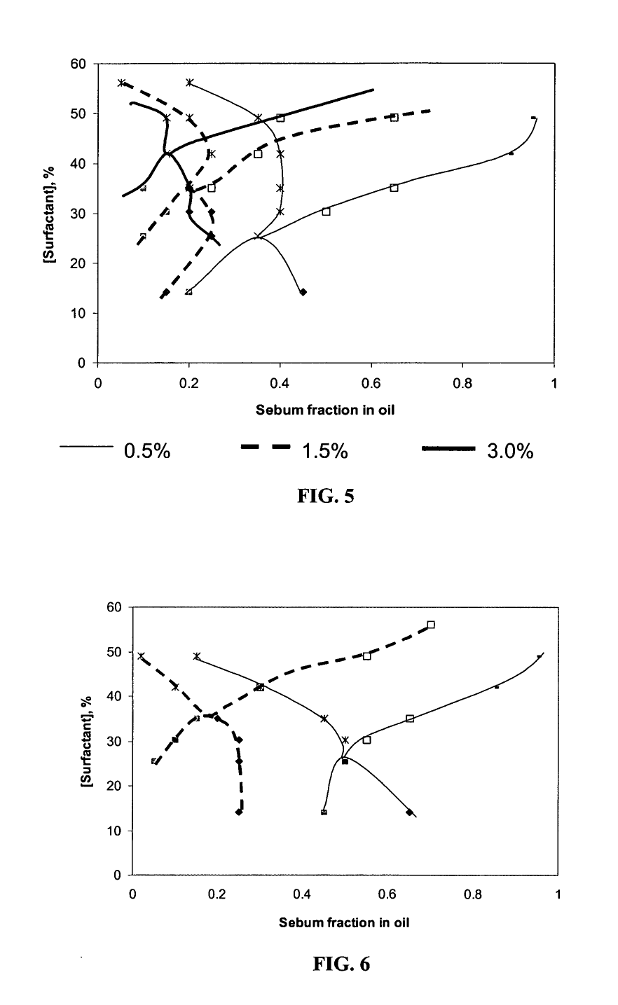

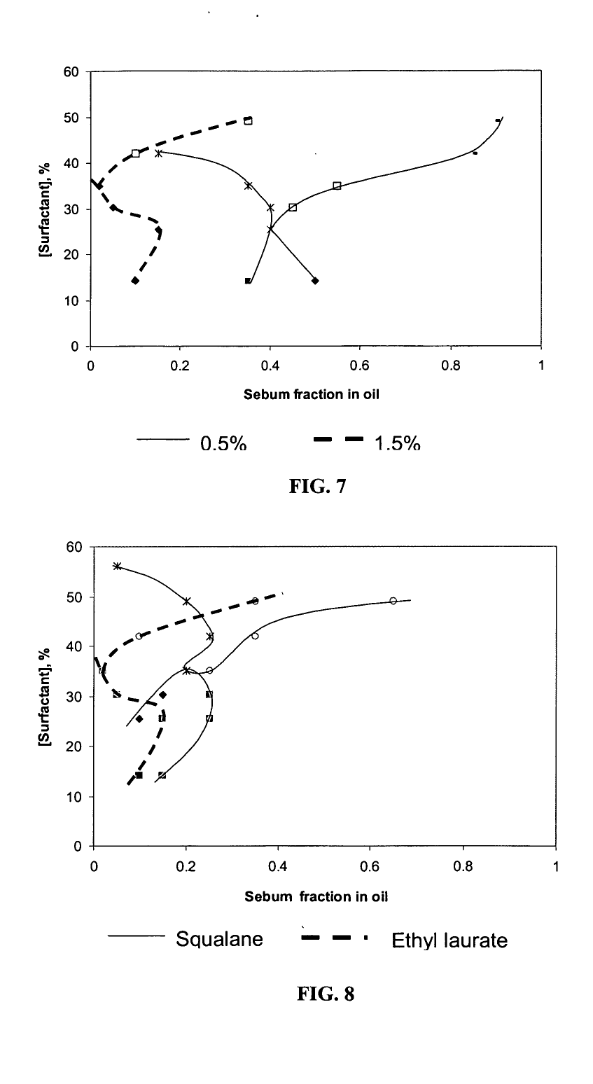

FIG. 5 shows the effect of salinity on phase behavior for the systems with squalane as co-oil. Both the body and the tail of the fish are observed for all three salinities: 0.5%, 1.5% and 3.0% NaCl. As mentioned, the phase behavior with squalane appears to be similar to that studied with squalene (FIG. 3). Briefly: (i) nonmicroemulsion is observed at low surfactant concentration and high sebum fraction in oil; (ii) a Winsor Type I-III-II transition occurs at low surfactant concentrations and a Winsor Type I-IV-I transition occurs at high surfactant concentrations when the sebum fraction in the oil increases; (iii) a Winsor Type I-IV-II transition appears at intermediate surfactant concentration. As salt concentration increases, the concentration at which Type IV forms, as denoted by “C,” increases whereas the fraction of sebum in oil or “F” at this point decreases. A similar trend is also seen for the system with IPM and EL, as shown in FIGS. 6 and 7. The values of C and F for the systems with different co-oil and different salinities are shown in Table 4.

Adding salt increases the hydrophobicity of the surfactant system. At a given co-oil type, an increase in salinity shifts the phase behavior from Type I-III-II. When the surfactant system becomes more hydrophobic, the surfactant system can microemulsify the more hydrophobic oil. This is consistent with the observation that when salt concentration increases from 0.5% to 3.0%, Type III microemulsion forms at lower sebum fraction in oil, which is more hydrophobic than the oil mixture with higher sebum fraction. The concentration “C” increases with increasing salinity. This can be explained by the fact that adding salt in general decreases the solubilization because the salt molecules adsorbed at the interface displace the surfactant molecules, and reduce the overall number of interaction per unit area (Bourrel and Schecter, 1988)). This behavior is also similar to the system when low molecular weight alcohol is used.

High salinity systems create Winsor Type I-III-II and I-IV-II transitions at low and high surfactant concentration, respectively. Neither nonmicroemulsion nor sponge phases is present. In addition, the fish seems to be more vertical than the fish at low salt concentration, indicating less partitioning of surfactants or linkers into the excess phases.

### Example 5

### Effect of the Type of Co-Oil on the Fish Diagram

The effect of the type of co-oil on the phase behavior can be considered in terms of the change in the concentration C and the fraction of sebum in oil F, as shown in Table 4. Changes in the C and F values at 0.5% NaCl are not significant when the hydrophilicity of the co-oil increases from squalene to ethyl laurate. However, a decrease in the F value at 1.5% NaCl is observed: the F values are 0.2, 0.175, and 0.02 for squalane, IPM, and EL, respectively. FIG. 8 shows the effect of the type of co-oil on the phase behavior at 1.5% NaCl. The comparison is made only between squalane and ethyl laurate due to the relatively clear difference between the two types of the co-oil. The result for ethyl laurate at 1.5% NaCl is of interest. The fish diagram appears at very low sebum fraction in oil, or the F value is very close to zero, as shown in FIG. 8. In other words, the phase behavior (Type II) is more robust over the entire range of the sebum fraction in oil, compared to the results with squalane.

This surfactant/linker system is relatively hydrophilic, creating the positive curvature with pure squalene at 0.5% NaCl, as shown in FIG. 3. The addition of 1.5% NaCl makes the system become more hydrophobic and helps decrease the curvature of the surfactant membrane. Using co-oil that has lower EACN such as ethyl laurate further decreases the curvature. As seen in FIG. 8, a Type III is observed in the absence of the sebum oil (the sebum fraction is zero). As the hydrophilicity of the oil increases by increasing the sebum fraction in oil, the interacting between surfactant and oil is enhanced. This leads to a negative curvature, and the Type II microemulsion is formed. For the system with squalane which is a more hydrophobic oil, compared to ethyl laurate, a higher fraction of sebum oil is required to obtain the same curvature to the curvature obtained from the system with ethyl laurate.

### Example 6

### Cleansing Formulation

The following Table 5 includes a non-limiting embodiment of a skin cleansing formulation of the present invention.

TABLE 5
Skin Cleansing Formulation*

| Ingredient | Grams | % of batch | % actives |
|---|---|---|---|
| AOT | 29.9 | 8.5 | 100 |
| Span 80 | 38.3 | 10.9 | 100 |
| Alkyl Glucoside | 50.4 | 14.4 | 75 |
| NaCl | 1.8 | 0.5 | 100 |
| Water | 192.8 | 55.1 | |
| Squalene | 36.8 | 10.5 | 100 |
| Total | 350 | 100 | |

*Preparation of formulation: AOT, Span 80, alkyl glucoside, NaCl, and water are weighed into a bottle, then stirred overnight using a magnetic stirrer. Co-oil is added after all components are mixed. The system subsequently becomes homogenous.

Derivatives of these ingredients can be used as substitutes. Additionally, other ingredients with similar physiological activities are contemplated as being useful as substitutes or as additional ingredients that can be used with the compositions of the present invention. Table 6 includes data concerning the cleansing efficacy of the composition in Table 5.

TABLE 6

| Cosmetic Soil | % of Soil Removed by Test Sample |
|---|---|
| Makeup Foundation A | 94.5 |
| Makeup Foundation B | 92.8 |
| Lipstick | 95.5 |
| Waterproof Mascara | 69.4 |
| Eye Shadow | 99.4 |

The procedure used for obtaining these results included: The baseline color of the test skin site is measured in terms of light/dark (L*) with the Minolta Chromameter CR-400. The cosmetic soil is applied, allowed to air dry, and the color re-measured. The difference between these two values is a measure of the amount of cosmetic present. The cleanser is then rubbed into the test site in a standard manner and then wiped off with a damp cloth. The color of the test site is again measured. The difference between this and the baseline value is a measure of the amount of color remaining on the skin. The efficacy of the cleanser is determined as a ratio of the amount of cosmetic soil removed versus the amount applied.

### Example 7

### Lipid Delivery Formulation

The following Table 7 includes a non-limiting embodiment of a skin cleansing formulation of the present invention.

TABLE 7
Formulation For Lipid Delivery to Skin Barrier*

| Ingredient | Grams | % of batch | % actives |
|---|---|---|---|
| AOT | 25.4 | 12.7 | 100 |
| Crill 6 | 32.5 | 16.3 | 100 |
| Alkyl Glucoside | 42.7 | 21.3 | 75 |
| Lipid Component | 20.0 | 10.0 | 100 |
| NaCl | 1.0 | 0.5 | 100 |
| Water | 78.4 | 39.2 | |
| Total | 200 | 100 | |

*Preparation of formulation: AOT, Crill 80, alkyl glucoside, NaCl, and water are weighed into a bottle, then stirred overnight using a magnetic stirrer. Co-oil is added after all components are mixed. The system subsequently becomes homogenous.

Derivatives of these ingredients can be used as substitutes. Additionally, other ingredients with similar physiological activities are contemplated as being useful as substitutes or as additional ingredients that can be used with the compositions of the present invention.

### Example 8

### Oil Control Formulation

The following Table 8 includes a non-limiting embodiment of an oil-control formulation for oily skin of the present invention.

TABLE 8*

| Ingredient | Grams | % of batch | % actives |
|---|---|---|---|
| AOT | 2.0 | 4.0 | 100 |
| Crill 6 | 2.0 | 4.0 | 100 |
| Decyl Glucocide | 8.2 | 16.3 | 49 |
| Cab-O-Sil | 2.5 | 5.0 | 100 |
| NaCl | 0.1 | 0.1 | 100 |
| Water | 35.3 | 70.6 | |
| Total | 50.0 | 100.0 | |

*Preparation of formulation:
AOT, Crill 80, Decyl glucoside, NaCl, and water are weighed into a bottle, then stirred overnight using a magnetic stirrer. Co-oil is added after all components are mixed. The system subsequently becomes homogenous.

Derivatives of these ingredients can be used as substitutes. Additionally, other ingredients with similar physiological activities are contemplated as being useful as substitutes or as additional ingredients that can be used with the compositions of the present invention.

### Example 9

### Phase Studies of Microemulsions

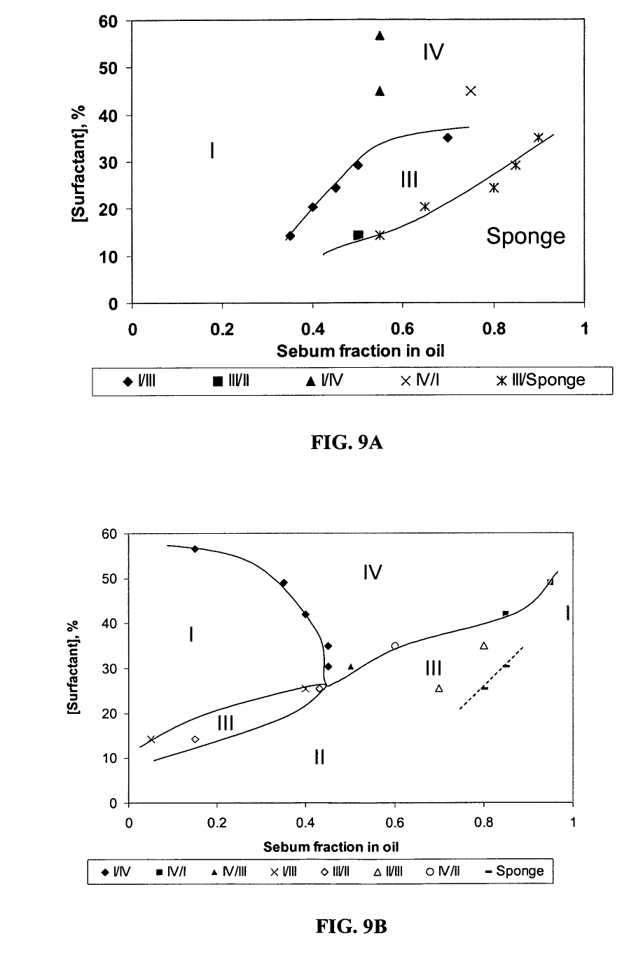

The inventors performed phase study experiments on non-limiting examples of microemulsions of the present invention. The Microemulsions in FIG. 9A-B included squalene, a [NaCl]=0.5%, AOT 4% wt., Span 80 5.13% wt, and AG 5.16% wt, and artificial sebum. The volume of oil equals the volume of surfactants in FIG. 9A. The volume of oil equaled the volume of water in FIG. 9B.

FIG. 9A shows that microemulsion Type I-III transition is observed when sebum fraction in oil or hydrophilicity increases (sebum is more hydrophilic than squalene). The closer the hydrophilicity of microemulsified oil to the surfactant used, the more likely the oil is solubilized in surfactant aggregates, resulting in a Winsor I-III-II transition. A Winsor II microemulsion occurs when the surfactant is in an oil phase. A Winsor Type II is observed for a small range of sebum fraction in the oil at the lowest surfactant concentration studied before a sponge phase is observed. A Winsor Type IV (one-phase) microemulsion is observed at high surfactant concentration. This occurs when the system contains enough surfactant to completely microemulsify the oil and the water.

FIG. 9B shows a fish diagram when WOR=1. The body of the fish appears at low surfactant concentrations (up to 25% surfactant) although the fish body is not vertical, indicating the partitioning of surfactant into excess phases. The tail of the fish occurs at surfactant concentrations higher than 25%. A Winsor Type I-III-II transition is observed when the sebum fraction in oil or hydrophilicity of the oil mixture increases in the low surfactant concentration regime. At high surfactant concentrations (higher than 40%), a Winsor Type I-IV-I transition appears. A Winsor Type IV microemulsion occurs at a lower surfactant concentration than the system shown in FIG. 9A due to the reduced amount of oil. This shows that a lower surfactant concentration is possible to microemulsify both oil and water.

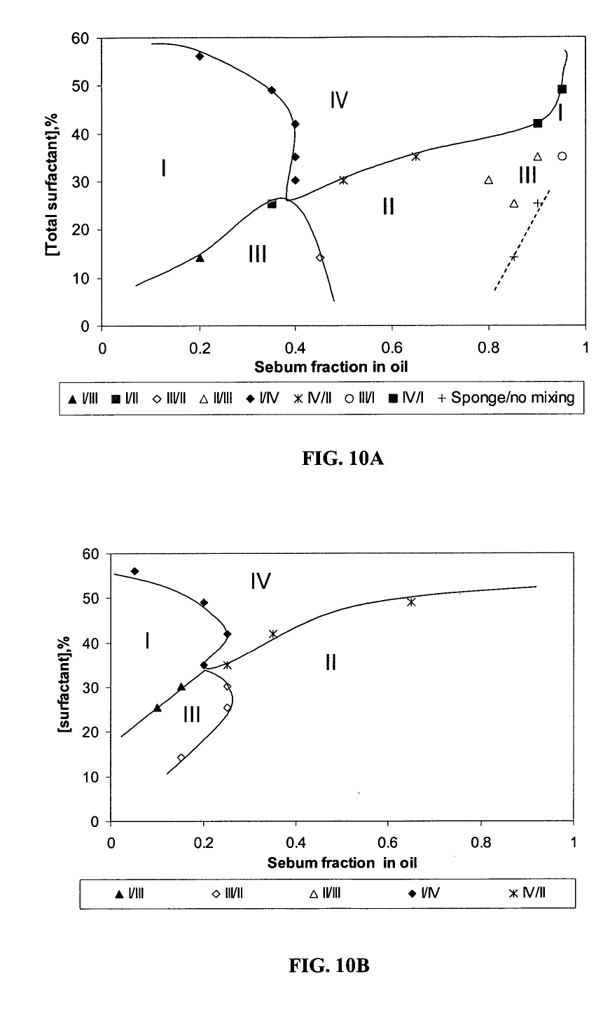

The microemulsions in FIG. 10A-B included squalane, a [NaCl]=0.5% (FIG. 10A) or [NaCl]=1.5% (FIG. 10B), AOT 4% wt., Span 80 5.13% wt, and AG 5.16% wt, and artificial sebum. The volume of oil equaled the volume of water. The data presented in FIGS. 10A-B shows that NaCl has an effect on the solubility of sebum in the oil phase of the microemulsion. The higher the salinity, the lower the fraction of sebum to oil is observed, thereby using more squalene. For example, FIG. 10A shows that the phase behavior with squalane is somewhat similar to the system with squalene except the body of the fish is wider for squalane. A Winsor Type I-III-II transition is observed at low surfactant concentrations, I-IV-II at intermediate concentration, and I-IV-I at high concentrations as sebum fraction in oil increases.

FIG. 10B shows that when the sebum fraction in oil increases, a Winsor I-III-II transition appears at surfactant concentrations up to 35%. At higher surfactant concentration, a Winsor I-IV-II transition is observed. In the presence of 1.5% NaCl, Winsor IV occurs at a higher surfactant concentration but lower sebum fraction in oil, compared to the system with 0.5% NaCl. This indicates that at high salinity, which causes an increase in hydrophobicity of the surfactant system, a greater amount of co-oil may be useful in order to match the HLB of the surfactant.

Table 9 shows that an amount of sebum oil in microliters (EL) that can be used to form a microemulsion (Type I, II, III, and IV) for 4 different formulations. This is done to observe the effect of surfactant concentration and the presence of co-oil on the efficiency of sebum microemulsification. The experiment was conducted by titrating the sebum oil into a 500 μl formulation with an increment of 100 μL sebum oil. For example, for formulation 1, a Winsor Type IV microemulsion was initially observed when the first 100 microliters of sebum oil was added. Then at the volume of 2760 μL of sebum oil, a Winsor Type II microemulsion is observed. For the second formulation, a Winsor Type IV was observed at added sebum oil volumes up to 3540 μL when 12.5% squalene is present This suggests that the microemulsification efficiency is greater in the presence of co-oil than in absence of co-oil.

TABLE 9

| Formulation | | | | | | | |
|---|---|---|---|---|---|---|---|
| (1-4) | [surfactant] % | [NaCl] % | % squalene | IV* | II* | I* | III* |
| 1 | 56.07 | 0.5 | 0 | 2660 | 2760-6140 | | |
| 2 | 49.07 | 0.5 | 12.75 | 3540 | 3640-8140 | | |
| 3 | 42 | 0.5 | 5.8 | 1100 | | 1200-2200 | 3200-5200 |
| 4 | 30.29 | 0.5 | 10.5 | 700 | 800-1050 | | 1300-4800 |

*Sebum Oil in microliters.

### Example 10

### Skin Critical Surface Tension (CST)

CST is a method of assessing the skin wettability quantitatively. CST was calculated using the Zisman equation, from the contact angle at equilibrium, of droplets of liquids whose surface tension was known. Zisman's equation is Cosθ=1-b(γ_{LA} γ_{C}) (applied to low energy aqueous solution and low energy/hydrophobic surface. Therefore only skin with CST<30 mN/n can be studied).

Results show a CST_{forearm}˜27.5 mN/m and CST_{forehead}>50.7 mN/m. Forehead has both sebum and sweat, which can form emulsion, increasing CST (become less hydrophobic). γ_{sebum} equaled 24 mN/m and γ_{sweat} equaled 40 mN/m. Because the forehead has high surface energy, Zisman's equation was not applied. Secretion of sebum and sweat contributes to an increased skin CST through an emulsion of sebum and sweat: W/O type for low sweating and O/W for high sweating. These two types of emulsion have been observed on the forehead before and after sweating. Cleansing with soap and rinsing decreased the CST, and therefore wettability of the skin. A much greater contact angle after the cleansing was observed, this was due to the sebum removal and surface hydration. Moisturizing the skin surface lowered the CST.

### Example 11

### Cleansing Formulation

The following Table 10 includes a non-limiting embodiment of a skin cleansing formulation of the present invention. The formulation is formulated as a transparent Type-IV alcohol-free microemulsion.

TABLE 10
Skin Cleansing Formulation*

| Ingredient | wt % |
|---|---|
| Olivem 300 (olive oil PEG-7 esters) | 3.04 |
| Diisopropyl adipate | 10.90 |
| Trivasol BW (PEG-8 caprylic/capric glycerides) | 27.34 |
| Isolan GI-34 (polyglyceryl-4-isostearate) | 7.28 |
| Isostearic acid | 6.24 |
| Squalane (2,6,10,15,19,23-Hexamethyltetracosane) | 6.50 |
| Water | 22.34 |
| Isododecane | 16.36 |
| Total | 100 |

*Preparation of formulation: All ingredients are weighed and placed into a bottle. The ingredients are subsequently stirred until a homogenous composition is obtained.

In non-limiting embodiments, the ingredients in the Table 10 formulation can have various functions. By way of example only: Olivem 300 can be used as a non-ionic surfactant or emulsifier; Diisopropyl adipate can function as a lipophilic linker; Trivasol BM can function as a non-ionic surfactant or emulsifier; Isolan GI-34 can function as a non-ionic surfactant or emulsifier or as a hydrophilic linker; Isostearic acid can function as a lipophilic linker; and Squalane and Isododecane can both function as co-oils. It is also contemplated that derivatives of these ingredients can be used as substitutes. Additionally, other ingredients with similar physiological activities are contemplated as being useful as substitutes or as additional ingredients that can be used with the compositions of the present invention.

In one non-limiting aspect, the Table 10 formulation was tested to determine its ability to spontaneously emulsify oil. Squalane was used as the testing oil (i.e., the oil to be spontaneously emulsified). In the procedure, an aliquot of the Table 10 formulation was obtained. Squalane in an amount of 10.0% of the weight of the aliquot was subsequently added to the aliquot. The Table 10 formulation spontaneously emulsified all of the testing oil. Further, the formulation remained transparent after the spontaneous emulsification.

All of the compositions and/or methods disclosed and claimed in this specification can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

U.S. Pat. No. 4,146,499

U.S. Pat. No. 4,568,480

U.S. Pat. No. 6,495,126

U.S. Pat. No. 6,495,126

Barany and Merrifield, In: The Peptides, Gross and Meienhofer (Eds.), Academic Press, NY, 1-284, 1979.

Acosta et al., J. Surfactants Deterg., 6:1-12, 2003.

Acosta et al., J. Surfactants Deterg., 5:151-157, 2002.

Acosta et al., Environ. Sci. Technol., 36:4618-4624, 2002.

Bourrel and Schecter, In: Microemulsions and Related Systems, Marcel Dekker, NY, 1988.

Daicic et al., Langmuir, 11:2451-2458, 1995.

Graciaa et al., Langmuir, 9(3):669-672, 1993.

Houghten et al., Infect. Immun., 48(3):735-740., 1985.

Huang and Lips, Langmuir, 20(9):3559-3563, 2004.

Merrifield, Science, 232(4748):341-347, 1986.

PCT Appln. PCT/EP02/05977

Remington's Pharmaceutical Sciences, 18th Ed. Mack Printing Company, 1990.

Rieger, In: Surfactants in Cosmetics, Surfactant Science Series, 1999.

Rosen, In: Surfactants and Interfacial Phenomena, 2^{nd} Ed., John Wiley & Sons, NY, 1:4-31,1988.

Salager et al., Soc. Petrol. Eng. J., 19:271-278, 1979.

Stewart and Young, In: Solid Phase Peptide Synthesis, 2d. ed., Pierce Chemical Co., 1984.

Tamn et al., J. Am. Chem. Soc., 105:6442, 1983.

Tungsubutra and Miller, J. Am. Oil Chem. Soc., 71:65-73, 1994.

Von Corswant et al., J. Pharm. Sci., 87:200-208, 1998a.

Von Corswant et al., Langmuir, 13:5061-5070, 1997.

Von Corswant et al., Langmuir, 14:3506-3511, 1998b.

Von Corswant et al., Langmuir, 14:6864-6870, 1998c.

1. A composition comprising an alcohol-free microemulsion, the microemulsion comprising:
(a) a surfactant; and
(b) a lipophilic or a hydrophilic linker,
wherein the composition is formulated as a cosmetic composition.

2. The composition of claim 1, wherein the composition is capable of spontaneously emulsifying a triglyceride.

3. The composition of claim 1, wherein the composition is capable of spontaneously emulsifying sebum.

4-5. (canceled)

6. The composition of claim 1, wherein the microemulsion is comprised in a cosmetic vehicle.

7. (canceled)

8. The composition of claim 1, wherein the microemulsion is a single bicontinuous phase of water and oil.

9. The composition of claim 8, wherein the microemulsion is transparent.

10. The composition of claim 1, wherein the microemulsion is an oil-in-water microemulsion.

11. The composition of claim 1, wherein the microemulsion is a water-in-oil microemulsion.

12. The composition of claim 1, wherein the microemulsion is a two phase system.

13-14. (canceled)

15. The composition of claim 1, wherein the microemulsion is a three phase microemulsion.

16-19. (canceled)

20. The composition of claim 1, wherein the surfactant is an anionic surfactant, a cationic surfactant, a nonionic surfactant, an amphoteric/zwitterionic surfactant, or mixtures thereof.

21-27. (canceled)

28. The composition of claim 1, wherein the lipophilic linker is a glycerol monooleate, monoglyceride, an alkyl sorbital ester, a polyoxyethylene derivative of a sorbitan ester, or sorbitan isosterate (Crill 6).

29-31. (canceled)

32. The composition of claim 1, wherein the hydrophilic linker is an alkyl glucoside, sodium mono or dimethyl naphthalene sulfonate (SMDMS), or sodium xylene sulfonate.

33-45. (canceled)

46. The composition of claim 1, wherein the composition comprises a lipophilic and a hydrophilic linker.

47. The composition of claim 46, wherein the composition comprises:
(a) from about 0.1% to about 50% of the surfactant;
(b) from about 0.1% to about 50% of the lipophilic linker; and
(c) from about 0.1% to about 50% of the hydrophilic linker.

48. The composition of claim 1, further comprising a co-oil.

49. The composition of claim 48, wherein the co-oil is squalene, squalane, isopropyl myristate, ethyl laurate, artificial sebum, a cosmetic ester comprising from about a C6 to about a C30 group, or a compound comprising an equivalent alkane carbon number (EACN) similar to sebum, a mineral oil, a vegetable oil, an animal oil, oleyl oleate, chloresterol, glycerol tricaprylate, mineral oil, olive oil, almond oil, caprylic triglyceride, oleyl eructate, coco caprylate/caprate, or dioctyl cyclohexane.

50-53. (canceled)

54. The composition of claim 1, further comprising a hydrotrope.

55. The composition of claim 54, wherein the hydrotrope is an alkyl glucoside, sodium mono or dimethyl naphthalene sulfonate (SMDMS), sodium xylene sulfonate, or ammonium xylene sulfonate.

56-63. (canceled)

64. The composition of claim 1, further comprising a salt.

65. The composition of claim 64, wherein the salt is NaCl, KCl, CaCl₂, or MgCl₂.

66. The composition of claim 1, further comprising an active ingredient.

67. The composition of claim 66, wherein the active ingredient is a vitamin, a mineral, a humectant, an emollient, an anti-oxidant, an oil, a lipid, a botanical, a tanning compound, a skin lightening compound, a UVA absorber, a UVB absorber, a sunscreen, an infrared reflector, or an infrared absorber.

68. A method of cleaning skin or hair comprising applying to the skin or hair a composition comprising an alcohol-free microemulsion, the microemulsion comprising:
(a) a surfactant; and
(b) a lipophilic or a hydrophilic linker,
wherein applying the composition cleans the skin or hair.

69. The method of claim 68, wherein the composition spontaneously emulsifies a triglyceride.

70. The method of claim 68, wherein the composition spontaneously emulsifies sebum.

71. (canceled)

72. The method of claim 68, wherein the composition spontaneously emulsifies a triglyceride, sebum, or oil that is on the skin or hair.

73-82. (canceled)

83. A method of delivering an active agent to skin or hair comprising applying a composition to the skin or hair, the composition comprising:
(a) an alcohol-free microemulsion, the microemulsion comprising:
(i) a surfactant; and
(ii) a lipophilic or a hydrophilic linker; and
(b) an active agent,
wherein applying the composition to the skin or hair delivers the active agent to the skin or hair.

84-93. (canceled)

94. A method of delaying the transmission of sebum through a cosmetic composition that is on skin comprising:
(a) applying a composition comprising an alcohol-free microemulsion to the skin, the microemulsion comprising:
(i) a surfactant;
(ii) a lipophilic or a hydrophilic linker; and
wherein applying the composition to the skin absorbs sebum from the skin, and
(b) applying a cosmetic composition to the skin,
wherein a reduction of sebum on the skin prior to topically applying the cosmetic composition delays the transmission of the sebum through the cosmetic composition that is subsequently applied to the skin.

95-98. (canceled)
